(19) United States
(12) Patent Application Publication
(10) Pub. No.: US 2015/0216871 A1
(43) Pub. Date: Aug. 6, 2015

(54) Use of a Humic Acid Preparation for Treating Warm-Blooded Animals

(71) Applicant: WH PHARMAWERK WEINBÖHLA GMBH, Weinböhla (DE)

(72) Inventors: Manfred Kühnert, Leipzig (DE); Monika Krüger, Leipzig (DE); Svent Haufe, Diera-Zehren (DE); Awad Sheata, Leipzig (DE)

(21) Appl. No.: 14/427,311

(22) PCT Filed: Sep. 11, 2013
(86) PCT No.: PCT/DE2013/100324

§ 371 Date: Mar. 11, 2015

(30) Foreign Application Priority Data
Sep. 11, 2012 (DE) 10 2012 108 438.6

(51) Int. Cl. A61K 31/538

(52) U.S. Cl. CPC A61K 31/538

(57) ABSTRACT

The invention relates to the use of a humic acid preparation RB4 or lignocellulose carbon isolate for treating diseases in warm-blooded animals cause by glyphosate herbicide, whereby the glyphosate herbicide is bound to the humic acid preparation and reduced the growth-inhibiting effect of glyphosate herbicide on physiological germs. The invention also related to the use of humic acid preparation RB4 or lignocellulose carbon isolate for external skin application so that it leads to the improvement or maintenance of skin health.

### CROSS-REFERENCE TO RELATED APPLICATIONS

This application is the U.S. national stage of International Application No. PCT/DE2013/100324 filed on Sep. 11, 2013, and claims the benefit thereof. The international application claims the benefit under 35 USC 119 of German Application No. DE 10 2012 108 438.6 filed on Sep. 11, 2012; all applications are incorporated by reference herein in their entirety.

### BACKGROUND

The invention relates to the use of humic acid preparation RB4 or lignocellulose carbon isolate for treating diseases in warm-blooded animals caused by glyphosate, especially if the herbicide is present in the animal feed.

The total herbicide glyphosate—commonly known under the brand name Roundup®—is a development from the 1970s. Since 1996 imported RR (Roundup Ready) soya beans are approved in Europe as animal feed and food item without marking obligation. The marking obligation came into force only in 2004 as “GMO” (Genetically Modified Organism). The active ingredient is universally applicable. It inactivates the enzyme 5-enolpyruvyl-shikimic acid-3-phosphate synthase (EPSPS) and thus prevents the synthesis of aromatic amino acids (phenylalanine, tyrosine, and tryptophan) in plants and some micro-organisms, which then die.

Some of these micro-organisms are also part of the autochthonous microflora and fauna and microbiota of the gastrointestinal tract of humans and animals. The reduction or total lack of the so-called health-promoting bacteria (e.g. lactobacilli, bacilli, enterococci) or their metabolic products lead to microbial imbalances in this organ system, which can lead to adverse health effects, because some of the micro-organisms such as Clostridium (C.) perfringens, C. tetani, C. botulinum, Salmonella Typhimurium are tolerant to resistant for glyphosate and it may result in diseases with these pathogens if the antagonistic autochthonous flora are absent.

EU threshold values for many animal feed and food items are indeed fixed, but they do not provide any information on the impact of chronic, often lifelong intake of the active substance on the organ systems of humans and animals. Although the daily compatible glyphosate intake postulated by EU is 0.3 mg/kg of body weight, the statements on its consequences on the nerve, hormone, metabolic and immune system of the warm-blooded animals is missing.

So far the following long-term effects glyphosate are described by various working groups: genotoxicity, carcinogenity, cytotoxicity and teratogenity as well as hormone reduction. Other effects of glyphosate in mammals are directed towards cytochrome p450, aromatase, oestrogen/testosterone receptors as well as the breathing, hepatic and reproduction system.

The chemical description of the herbicide and its derivatives is known. It is used for treating agricultural crops to protect them from pests and weeds. Here there is a wide-area application without any special protective measures and at the same time unwanted influence of quality of raw harvest products and the resultant fodder and food items through the application of herbicide. Adverse health effects in humans and animals, reduction of food, fodder and raw product qualities and subsequent damages in the food chain are described.

In particular, the concentrations absorbed in the bodies of warm-blooded animals are excreted through the waste products of metabolism and also affect the nutrient cycle, which in turn leas to sustained damages to the micro-organisms in soil and in mammals. The absorption into the nutrient cycle leads to a destructive intervention in the equilibrium of plant health and to a strain on the natural cycle of substances in nature.

The current literature does not have test reports, which describe a method or something similar for the deactivation of substance group to which glyphosate belongs. A method or process, which prevents or stops the aforementioned effects especially in warm-blooded animals or its autochthonous microflora and fauna or microbiota, is not known according to the current state of technology.

Humic acids are known according to prior art. The publication DE 196 50 317 C2 describes lignocellulose carbon isolate CLK-GA, also known as leonardite, which of course contains humic acid as its main component. They are those amorphous portions of black-brown to brown coloured substances in soil, lignites and peats, which result from the so-called humification of the rotting organic material. They are soluble in diluted based and insoluble in mineral acids. Natural (native) humic acids cannot be classified as clearly defined substances in terms of classical structural chemistry. It finds its fallout in that only nutrition models for chemical structures exist. Apart from cellulose and lignite structures, even polyionic structures with e.g. carbon acid ester, phenolic, hydroxyl, carbonyl and carboxyl groups belong to the building blocks. Even quinoid and flavonoid structures are present.

In particular, the humic acids resulting from plant products, to which lignite humic acids also belong, also have flavone structures (fisetin, quercetin, flavone, xanthine) in addition to their aromatic-phenolic basic composition.

Alkali-huminate-humic acid mixtures are used as source material for obtaining CLK isolation according to the invention as they are available, for example in accordance with the information in publication DD 44 155, from natural substances containing adequate quantity of humic substances. Furthermore, the humin-containing natural substances are supposed to have least amount of compounds hazardous to humans and animals. Such compounds can be acute or chronic toxic as well as genotoxic, teratogenic, mutagenic or carcinogenic substances. Humic acids of the previously described type are physically-chemically prepared natural humic acids, which are obtained from a specially selected lignite deposits, preferably lignites from deposits that do not have or have very little hazardous compounds mentioned above. In addition, even peats, moor and sea sediments can be used.

### SUMMARY

The invention relates to the use of a humic acid preparation RB4 or lignocellulose carbon isolate for treating diseases in warm-blooded animals cause by glyphosate herbicide, whereby the glyphosate herbicide is bound to the humic acid preparation and reduced the growth-inhibiting effect of glyphosate herbicide on physiological germs. The invention also related to the use of humic acid preparation RB4 or lignocellulose carbon isolate for external skin application so that it leads to the improvement or maintenance of skin health.

### DETAILED DESCRIPTION

Therefore, the objective of this invention is to reduce or to neutralise the effect of glyphosate and its derivatives found in animal feed in body, metabolic waste product and in soil, above all in the stomach-intestine microbiosis and on other body systems in order to maintain the microbiological activity in body, in soil and in the treatment of metabolic waste products of all types.

This objective is resolved by using a humic acid preparation for treating the diseases caused by an herbicide especially in warm-blooded animals, especially if the herbicide is present in animal feed products, whereby the herbicide is bound to the humic acid preparation and/or by reducing the growth-inhibiting effect of the herbicide on physiological germs.

The indicator germs Enterococcus faecalis and Bacillus badius were used to prove the glyphosate neutralising effect of CLK-GA. The pathogen Enterococcus faecalis was isolated from the microalgae Chlorella vulgaris to citrate-acid-tween-carbonate (CATC) agar. Suspicious colonies (gram positive, catalase negative cocci) were inoculated to blood agar plates (oxoid) and differentiated by using biochemical tests. The species differentiation was confirmed by means of MALDI-TOF-MS. The isolation of the pathogen Bacillus badius was done from Chlorella vulgaris to blood agar (oxoid). The species differentiation was done based on biochemical parameters. The confirmation was done by means of MALDI-TOF-MS.

Commercially available glyphosate preparation Roundup UltraMax® (450 g/l glyphosate as isopropylamine salt on SL formulation) was used for the test.

The value for the minimal inhibitory concentration of glyphosate for the indicator germs (MIC) was determined by using micro dilution method (MID) in accordance with the current specifications of the National Committee for Clinical Laboratory Standards (NCCLS). It was carried out for E. faecalis and B. Badius in RCM Bouillon (SIFIN).

The determination of the neutralising effect of humic acid product CLK-GA for glyphosate was done through glyphosate MIC. The MIC determinations for glyphosate and glyphosate-CLK-GA mixture was carried out in RCM Bouillon as described above. CLK-GA was used in the concentrations of 1.0, 0.5 and 0.25 mg/ml. The neutralisation of glyphosate was carried out in RCM Bouillon in the concentrations of 300 μg/ml, 600 μg/ml, 1200 μg/ml and 2400 μg/ml mixed with 1 mg/ml, 0.5 mg/ml and 0.25 mg/ml CLK-GA and kept overnight at 37° C. After centrifugation (3000 U/min) and filtration of the individual formulations through a centrifugal filter (3Kda, VWR, Mexico)m 104/ml KbE E. faecalis and B. Badius was given to these. After growing it overnight at 37° C., the contents of each tube was spread on CATC agar or blood agar and was evaluated after 48 h.

The tests showed the following results: The MIC of E. faecalis is 200 μg/ml and the MIC value for B. badius is 150 μg/ml of glyphosate. The MIC values of glyphosate and glyphosate-CLK-GA mixtures for E. faecalis and B. badius are presented in table 1. These values were increased through the humic acid product CLK-GA in the concentration of 1 mg/ml to 2400 μg/ml for both bacteria species. Glyphosate concentrations of 600 μg/ml were neutralised even with 0.5 mg/ml and 0.25 mg/ml CLK-GA. The addition of a glyphosate-CLK-GA mixture with an end concentration of 300 μg/ml glyphosate and 1 mg/ml of CLK-GA to 10⁴KbE/ml enterococci and bacillus suspension did not lead to elimination of germs in both the pathogen suspensions. The growth was absent in the glyphosate tests and the CKL-GA growth tests without glyphosate showed similar germ count like glyphosate-CLK-GA mixture.

The effects and advantages according to the invention are generally observed in the application of humic acid preparations, however they are very significant when a lignocellulose carbon isolate (CLK-GA) is used as humic acid preparation, which was obtained according to the above description (cf. publication DE 196 50 317 C2).

Table 1 shows the MIC values of glyphosate and glyphosate-CLK-GA mixture for Enterococcus faecalis and Bacillus badius.

| Indicator | MIC values | |
|---|---|---|
| germs | Glyphosate | Glyphosate-CLK-GA mixture |
| E .fecalis | 200 μg/ml | 2400 μg/ml |
| Bacillus | 150 μg/ml | 2400 μg/ml |
| badius | | |

The MIC values for E. faecalis increase in glyphosate-CLK-GA mixtures from 200 μg/ml to 2400 μg/ml (12 times) and for B. badius from 150 μg/ml to 2400 μg/ml (16 times). The binding of glyphosate to humic acid product CLK-GA depends on the concentration.

The effects are especially significant, if the herbicide is a total herbicide from the group of phosphanate or its derivatives, especially glyphosate. This was shown with the aforementioned tests. In particular, glyphosate is used widely.

It is even more advantageous, if a lignocellulose carbon isolate is used as humic acid preparation, because its production is already established and is cost-effective.

It is observed to be very effective, if the humic acid preparation is planned as oral intake that it leads to improvement or maintenance of the healthy status of the gastrointestinal tract (mucous membrane). For example, this effect occurs in ruminants, pigs, poultry and other warm-blooded animals. A generally bad general conditions of the old and young animals of cattle and bad milk quality improved so much just few weeks after the addition of humic acid preparation to the fodder that a normal health condition and a normal milk quality was observed. In particular, even the quality of food, which comes from stressed out mammals—milk for example—, has improved.

The same is applicable for human diseases, which can be attributed to the consumption of contaminated food. However, an application is also planned for prophylaxis, metaphylaxis, treatment and regeneration in human and veterinary medicine. This will ensure the maintenance of microbiological activity in body, which is impaired by the adverse effects of herbicides, especially glyphosate.

In case of alternative form of use, the humic acid preparation in this manner is planned for external application on skin so that it leads to improvement or maintenance of health. In particular, this effect is observed in a warm-blooded organism afflicted by glyphosate herbicide.

Other advantages have been observed if the humic acid preparation is used for cosmetic applications or is added to the cosmetic products.

A specific advantage was observed if the humic acid preparation is used in the manufacturing of food and fodder to improve the raw materials and the herbicide is bound to the humic acid preparation. Thus the parties contaminated with herbicides can be given fodder or food without fearing consequences. The quality of food, fodder and raw materials is increased. Application at this stage of the food chain is very beneficial, because we can prevent subsequent damages in the food chain.

Another benefit is due to the fact the humic acid preparation is added to the metabolic waste products or its subsequent products or processing steps, and the herbicide is bound to the humic acid preparation. As metabolic waste products of livestock are generally excreted as dung into the soil, it is contaminated by the herbicides reaching into blood circulation through food. This contamination endangers soil, because the micro-organisms responsible for the processes in soil, for example humus formation as important factor of soil fertility, are disturbed. However, if the unwanted herbicide is “′neutralised” with regard to its effect by binding it through the humic acid in the waste products, this adverse effect is prevented resulting in healthy and natural development of the soil.

The effect of waste products, for example liquid manure, on the soil life corresponds to that of the subsequent products, for example liquid manure treated in a biogas plant, with reference to the amount of herbicides reaching blood circulation. Therefore, in the interests of a high-quality dung product even biogas manure should be subject to treatment with humic acid preparation.

However, the treatment should take place before the waste products reach the biogas plant. Even there the sensitive population of micro-organisms can be disturbed by the influence of herbicides and thus the function, productivity and stability of the biogas plant can be impaired. This will be prevented through an advance treatment of waste products with humic acid preparation in accordance with the invention.

As an alternative, the humic acid preparation is applied to the contaminated soil and the herbicide is bound to the humic acid preparation. The effect on the soil life corresponds to the aforementioned effects, wherein an improvement can be expected even in the already damaged soil.

### BRIEF DESCRIPTION OF THE DRAWINGS

Further details, features and advantages of the invention can be taken from the following description of embodiment examples with reference to the associated drawings. It shows:

FIG. 1: Concentration-dependent neutralisation of glyphosate through humic acid product CLK-GA in the indicator system E. faecalis;

FIG. 2: Concentration-dependent neutralisation of glyphosate through the humic acid product CLK-GA in the indicator system B. badius; and

FIG. 3: Result of the cultivation of Enterococci suspensions (104/ml) after cultivation with filtrates of 600 μg/ml glyphosate—1 mg/ml CLK-GA mixture in the final concentration of 300 μg glyphosate/ml.

### DETAILED DESCRIPTION OF THE PREFERRED EMBODIMENTS

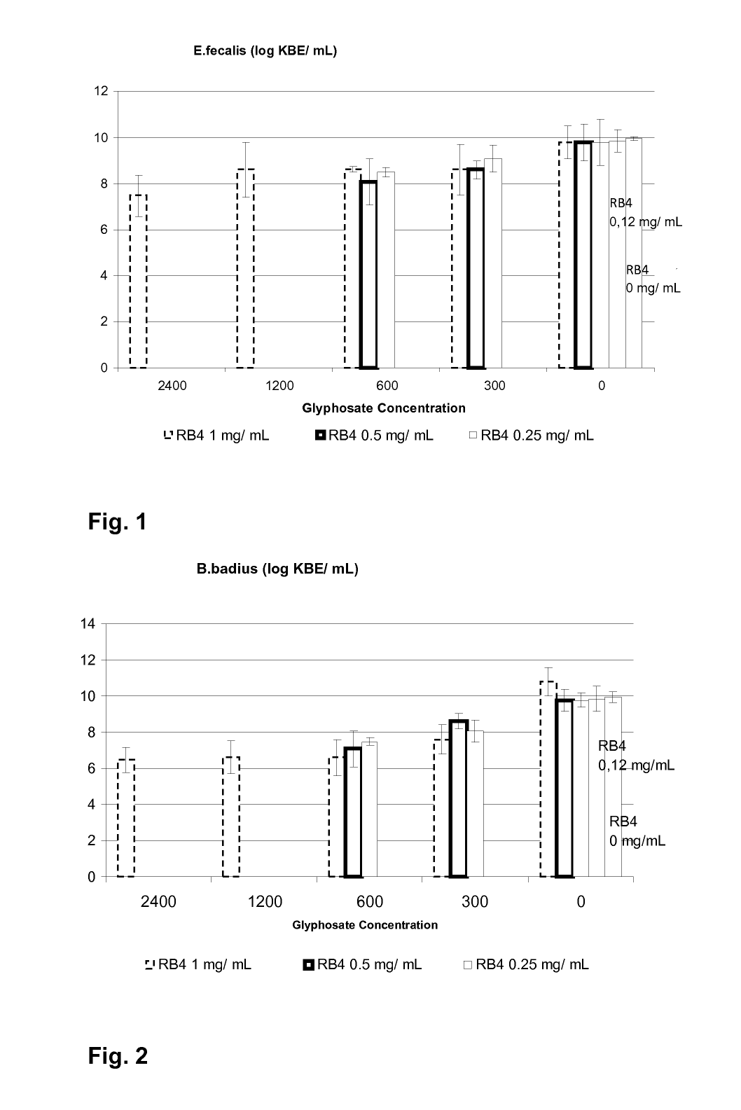

FIG. 1 shows the concentration-dependent neutralisation of glyphosate through humic acid product CLK-GA in the indicator system E. faecalis and FIG. 2 shows the concentration-dependent neutralisation of glyphosate through humic acid product CKL-GA in the indicator system B. badius. The indication CLK-GA 0.12 mg/ml in the “glyphosate 0 mg/ml” group of FIG. 1 refers to the previous column and the indication CLK-GA 0 mg/ml refers to the last column. In FIG. 2 the indication CLK-GA 0.12 mg/ml refers to the last but one column and the indication CLK-GA 0 mg/ml to the last column of the group “glyphosate concentration 0”.

The value for minimal inhibitory concentration (MIC) of E. faecalis is 200 μg/ml and the MIC value for B. badius is 150 μg/ml glyphosate. These values were increased by the humic acid product CLK-GA in the concentration from 1 mg/ml to 2400 μg/ml for both species of bacteria. Glyphosate concentrations of 600 μg/ml were neutralised with 0.5 mg/ml and 0.25 mg/ml of CLK-GA.

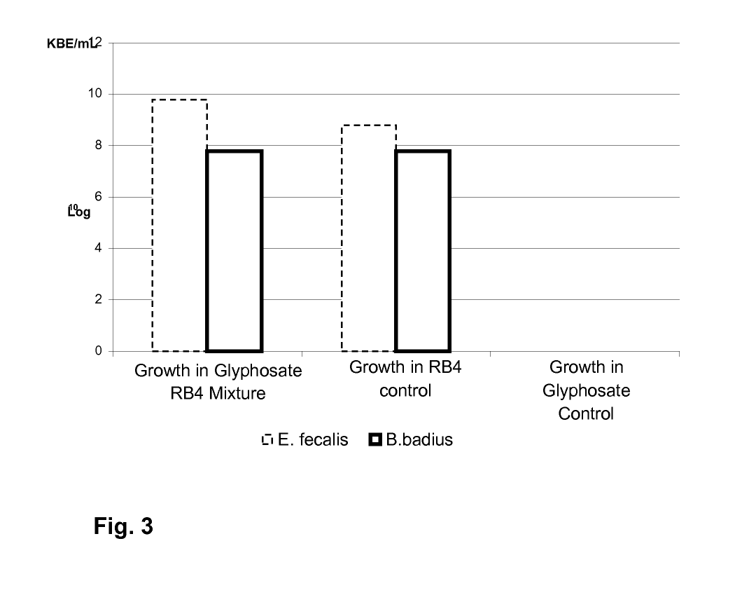

FIG. 3 shows the result of cultivation of Enterococci suspensions (104/ml) after cultivation with filtrates of a 600 μg/ml glyphosate—1 mg/ml CLK-GA mixture in the final concentration of 300 μg glyphosate/ml. The addition of a glyphosate CLK-GA mixture with a final concentration of 300 μg/ml glyphosate and 1 mg/ml CLK-GA WH67® to a 104 KbE/ml Enterococci or Bacillus suspension did not result in elimination of pathogens in both the pathogen suspensions.

The growth was absent in glyphosate tests and the CLK-GA growth tests (without glyphosate) showed similar pathogen count like glyphosate-CLK-GA mixture.

Laboratory tests show that the neutralisation effect of CLK-GA for the anti-microbial effect of glyphosate on E. faecalis was evident in glyphosate CLK-GA WH67® mixtures (1 mg/ml CLK-GA and different concentrations of glyphosate).

For an in-vivo application with CLK-GA in a severely impaired dairy farm through feed contaminated by glyphosate, the following initial condition was observed:
bad general condition of adult and young animals
bad conditions of the claws in spite of regular
bad fertility
very restricted milk quality (Salmonella count>70)
external condition of the animals is very dirty and shaggy
young adults are susceptible to diarrhoea
loss of appetite and diarrhoea
death events and
neurological and nervous deficiencies.

The treatment was done by mixing CLK-GA into animal feed over 4-6 weeks.

The following clinical events were observed:
the animals eat normally again,
the external condition became better (smooth and shiny coat),
improved claw health and care intervals became longer,
young and old animals no longer had diarrhoea,
no death events,
normalised social behaviour,
no neurological or nervous deficiencies were observed
Salmonella count<5.

Laboratory tests showed normal liver values.

### LIST OF REFERENCE NUMERALS

1 Growth in glyphosate CLK-GA mixture
2 Growth in CLK-GA test
3 Growth in glyphosate test

1. Use of humic acid preparation RB4 or lignocellulose carbon isolate for treating diseases in warm-blooded animal characterised in that the diseases are caused by a glyphosate herbicide, whereby the herbicide glyphosate is bound to the humic acid preparation and/or reduces the growth-inhibiting effect the glyphosate herbicide on physiological germs.

2. Use according to claim 1, whereby the glyphosate herbicide is present in food and animal feed products and the humic acid preparation is designed for oral intake that leads to the improvement or maintenance of healthy status of the gastrointestinal tract.

3. Use according to claim 1, whereby the glyphosate herbicide is present in food and animal feed products and the humic acid preparation is used in the production of food and animal food to improve the quality of the raw materials and the already present glyphosate herbicide is bound to the humic acid preparation.

4. Use according to claim 1, whereby the humic acid preparation is added to the metabolic waste products or subsequent products and glyphosate herbicide is bound to the humic acid preparation.

5. Use according to claim 1, whereby the humic acid preparation is applied to contaminated soil and the glyphosate herbicide is bound to the humic acid preparation.

6. Use of humic acid preparation RB4 or lignocellulose carbon isolate characterised in that the humic acid preparation is meant for external application on skin, which leads to the improvement or maintenance of skin health in a warm-blooded organism contaminated with glyphosate herbicide.

7. Use according to claim 6, whereby the humic acid preparation is used for cosmetic treatments or in cosmetic products.
